(19) United States
(12) Patent Application Publication
(10) Pub. No.: US 2004/0001798 A1
(43) Pub. Date: Jan. 1, 2004

(54) Self-adhesive cationic or amphoteric polyurethanes

(76) Inventors: Beatrice Perron, Jouy En Josas (FR); Serge Restle, Saint Prix (FR); Nathalie Mougin, Paris (FR)

Correspondence Address: NIXON & VANDERHYE, PC, 1100 N GLEBE ROAD, 8TH FLOOR, ARLINGTON, VA, 22201-4714, US

(21) Appl. No.: 10/323,853

(22) Filed: Dec. 20, 2002

(30) Foreign Application Priority Data
Dec. 20, 2001 (FR) FR 0116598

(51) Int. Cl. A61K 7/06; A61K 7/11; C08G 18/32; C08G 18/60

(52) U.S. Cl. 424/70.17; 528/68

(57) ABSTRACT

The invention relates to cationic or amphoteric polyurethanes bearing at least one tertiary or quaternary amine function and having a self-adhesion value—expressed by the maximum tensile force (Fₘₐₓ (in N)) recorded during the detachment by traction of two circular surfaces of 0.95 cm², coated with the said polyurethanes—of greater than or equal to 11 N. The invention also relates to the use of these self-adhesive cationic polyurethanes, in cosmetics and in particular in styling compositions.

The present invention relates to novel self-adhesive cationic or amphoteric polyurethanes, to the use of these self-adhesive polyurethanes in cosmetics, and also to cosmetic compositions, and in particular styling compositions, containing them.

Water-soluble cationic polymers, such as polymers based on dimethyldiallylammonium chloride, have been used for a long time in cosmetics, and in particular in haircare. The reason for this is that their good affinity (substantivity) for keratin substrates, and in particular their capacity to form continuous films around hairs, make them excellent candidates for protecting, enhancing and strengthening the hair.

However, on account of their high viscosity and their incompatibility with the majority of propellants, the polymers of this family are difficult to use in aerosol products such as lacquers.

The cationic polymers commonly used in haircare moreover have low self-adhesion, i.e. hair fibres surrounded with a coat of these cationic polymers adhere very little or not at all to each other.

The Applicant has discovered a novel family of particular cationic or amphoteric polyurethanes with a high level of self-adhesion, which have sufficient substantivity and very good styling power. This combination of properties makes them particularly suitable for use in rinse-out styling compositions such as styling shampoos.

Needless to say, their use in leave-in styling products is also advantageous since these self-adhesive cationic or amphoteric polyurethanes may then be used in markedly smaller amounts than the known cationic or amphoteric polymers or anionic or neutral self-adhesive polymers. The possibility of using the polymers of the present invention in small amounts facilitates their formulation and reduces the viscosity of the compositions obtained.

The self-adhesive cationic or amphoteric polyurethanes of the present invention may also be used in cosmetic fields other than that of styling. Thus, the introduction of small amounts of these polyurethanes into the majority of makeup products ensures good adhesion of the cosmetic deposits to the skin and gives them good cohesiveness and suppleness. The makeup does not crack and does not make the users' skin taut.

One subject of the invention is, consequently, cationic or amphoteric polyurethanes bearing at least one tertiary or quaternary amine function and having a self-adhesion value—expressed by the maximum tensile force (Fₘₐₓ (in N)) recorded during the detachment by traction of two circular surfaces of 0.95 cm², coated with the said polyurethanes—of greater than or equal to 11 N.

A subject of the invention is also cosmetic compositions, and in particular styling compositions, containing, in a cosmetically acceptable medium, at least one such self-adhesive cationic or amphoteric polyurethane.

A subject of the invention is also the cosmetic use of the novel self-adhesive cationic or amphoteric polyurethanes described above.

Finally, a subject of the invention is a process for treating keratin materials, comprising the application to the keratin materials of a cosmetic composition as described above, and also a styling process comprising the application of such a cosmetic composition to the hair, rinsing the hair and then shaping and drying the rinsed hair.

The self-adhesive nature of the cationic or amphoteric polyurethanes of the present invention is assessed according to the following protocol:

40 μl of an aqueous solution or dispersion containing 10% by weight of test polymer are deposited on the surface of two circular frosted-glass plates, each having a surface area of 0.95 cm² (11 mm diameter). The plates are left to dry for 48 hours at ambient pressure, at a relative humidity of 55% and at a temperature of 22° C.

The two plates are mounted in a machine for measuring tensile strength (Lloyd LR5K) and are pressed together for 20 seconds with a force of 3 N. The two plates are then separated, under the same temperature and relative humidity conditions, for 30 seconds by imposing a traction speed of 20 mm/minute, and the force required for this displacement, and more particularly the maximum force (Fₘₐₓ) in newtons (N) measured at the time of the sudden separation of the two polymer-coated surfaces, is recorded. Obviously, the self-adhesion of the polymers of the present invention is proportionately greater the larger the maximum force recorded.

In one preferred embodiment of the present invention, the cationic polyurethanes of the present invention comprise

(a) units derived from one or more tertiary or quaternary amines comprising two reactive functions containing labile hydrogen,

(b) units derived from one or more nonionic polymers comprising two reactive functions containing labile hydrogen, and

(c) units derived from one or more diisocyanates.

The tertiary or quaternary amines forming the cationic units (a) are preferably chosen from compounds corresponding to one of the following formulae:

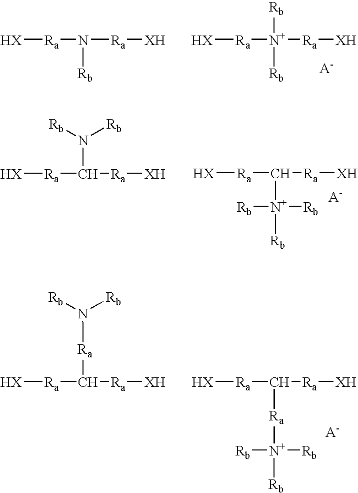

in which

each radical Rₐ represents, independently, a linear or branched C₁₋₆ alkylene, C₃₋₆ cycloalkylene or arylene group, all possibly being substituted with one or more halogen atoms and comprising one or more hetero atoms chosen from O, N, P and S,

each R_{b} represents, independently, a C₁₋₆ alkyl, C₃₋₆ cycloalkyl or aryl group, all possibly being substituted with one or more halogen atoms and comprising one or more hetero atoms chosen from O, N, P and S,

each radical X represents, independently, an oxygen or sulphur atom or a group NH or NR_{c}, in which R_{c} represents a C₁₋₆ alkyl group and

A⁻ represents a physiologically acceptable counterion.

As examples of tertiary amines that are particularly preferred for obtaining the self-adhesive cationic or amphoteric polyurethanes of the present invention, mention may be made of N-methyldiethanolamine and N-tert-butyldiethanolamine. These amines are preferably partially or totally neutralized with mineral or organic acids such as hydrochloric acid or citric acid.

The self-adhesive polyurethanes of the present invention may also comprise anionic units (d) derived, for example, from carboxylic or sulphonic acids comprising two functions containing labile hydrogen, such as dimethylolpropionic acid.

The self-adhesive polyurethanes of the present invention may also comprise nonionic monomer units (e) derived from nonionic monomer compounds comprising two functions containing labile hydrogen, such as butanediol or neopentyl glycol.

In one embodiment of the present invention, the self-adhesive polyurethanes of the present invention are cationic self-adhesive polyurethanes containing no units (d) and (e), and which consist essentially

(a) of units derived from one or more tertiary or quaternary amines comprising two reactive functions containing labile hydrogen,

(b) of units derived from one or more nonionic polymers comprising two reactive functions containing labile hydrogen, and

(c) of units derived from one or more diisocyanates.

The Applicant has found that the cationic polyurethanes have particularly advantageous self-adhesion properties when the polymer(s) forming the units (b) of the self-adhesive polyurethanes of the present invention have a glass transition temperature (Tg), determined by differential calorimetric analysis, of less than 0° C., preferably less than −5° C. and better still less than −10° C.

Examples of nonionic polymers capable of forming the units (b) that may be indicated include polyethers, polyesters, polysiloxanes, copolymers of ethylene and of butylene, polycarbonates and fluorinated polymers having a glass transition temperature of less than 0° C.

Polyethers are most particularly preferred, and among these poly(tetramethylene oxide).

These polymers preferably have a weight-average molar mass of between 400 and 10 000 and more particularly between 500 and 5 000.

The number of cationic charges borne by the self-adhesive polyurethanes of the present invention depends directly on the molar or weight ratio of the units (a) to the units (b). Needless to say, the units (c) are used in virtually equimolar amount relative to the sum of the units (a) and (b).

The molar ratio of the units (a) to the units (b) of the polyurethanes of the present invention is preferably between 0.01 and 50, more particularly between 0.1 and 6, better still between 0.2 and 5 and ideally between 0.3 and 5.

The diisocyanates forming the units (c) include aliphatic, alicyclic and aromatic diisocyanates.

Preferred diisocyanates are chosen from tetramethylxylylene diisocyanate, methylenediphenyl diisocyanate, methylenecyclohexane diisocyanate, isophorone diisocyanate, toluene diisocyanate, naphthalene diisocyanate, butane diisocyanate and hexyl diisocyanate. Needless to say, these diisocyanates may be used alone or in the form of a mixture of two or more diisocyanates.

An important parameter for selecting, among the cationic or amphoteric polyurethanes, those that have advantageous self-adhesion properties, is the glass transition temperature (Tg) of the final cationic polyurethane.

Specifically, the self-adhesive cationic or amphoteric polyurethanes preferably have at least one glass transition temperature of less than room temperature (20° C.), i.e. at room temperature, some of the polymer is in rubbery form rather than in vitreous form. The self-adhesion characteristics of the polymers of the present invention are particularly advantageous when the glass transition temperature is less than 0° C. and in particular less than −20° C.

The self-adhesive cationic or amphoteric polyurethanes of the present invention may also have several glass transition temperatures. When this is the case, the above indications regarding the glass transition temperature relate to the lowest glass transition temperature of the polymer.

The glass transition temperature of the polymers of the present invention is measured by Differential Scanning Calorimetry (DSC) under the following conditions:

To measure the glass transition temperature, a film about 150 mm thick of test polymer is prepared by depositing an aqueous solution or dispersion of the polymer in a circular Teflon die 40 mm in diameter and leaving the deposit to dry. The film is dried in an oven at a temperature of about 23° C. under a relative humidity of 45%, until the weight no longer changes. About 5 to 15 mg of the film are taken up and placed in a crucible, which is then introduced into the analyser. The thermal analyser is a DSC-2920 model from the company TA Instruments. The initial and final temperatures of the temperature sweep are chosen so as to surround the desired glass transition temperature. The temperature sweep is performed at a rate of 10° C./minute.

This analysis is performed according to ASTM standard D 3418-97 apart from the above changes.

The self-adhesive polyurethanes of the present invention are prepared according to known polycondensation methods. These methods are described especially in the following publications:

60 Years of PUR—J. E. Kresta, E. W. Eldred Ed. Technomic Publishing, 1998,

Waterborne and Solvent Based Surface Coating Resins and Their Application, Surface Coating Technology Series, Vol. 3, Polyurethanes, Paul Thomas, Wiley and Sons, 1998.

The self-adhesive polyurethanes of the present invention may be in the form of aqueous or oily solutions or dispersions.

As indicated above, the self-adhesive cationic or amphoteric polyurethanes of the present invention may be used in cosmetics in the form of care or makeup compositions for the skin or the integuments, in particular in the form of care, conditioning, makeup or fixing compositions for human keratin materials such as the hair and the eyelashes.

Preferably, these cosmetic compositions contain, in a cosmetically acceptable aqueous medium, from 0.01% to 40%, in particular from 0.05% to 20% and ideally from 0.1% to 10% by weight, of at least one self-adhesive cationic polyurethane of the present invention.

In one preferred embodiment of the present invention, the cosmetic compositions are styling compositions, and in particular rinse-out styling compositions, especially styling shampoos.

The cosmetically acceptable aqueous medium may contain various adjuvants and solvents commonly used in cosmetics, such as surfactants, anionic, amphoteric, zwitterionic or nonionic polymers, cationic polymers other than the cationic polyurethanes of the present invention, nacreous agents and/or opacifiers, organic solvents, fragrances, mineral, plant and/or synthetic oils, fatty acid esters, pigments and colorants, silicones, mineral or organic particles, pH stabilizers, preserving agents and UV absorbers.

The surfactants that may be used in the composition according to the present invention may be anionic, nonionic, amphoteric or cationic surfactants, or mixtures thereof.

Among the anionic surfactants that may be used, alone or as mixtures, in the context of the present invention, mention may be made especially of salts, and in particular alkali metal salts such as sodium salts, ammonium salts, amine salts, amino alcohol salts or alkaline-earth metal salts, for example magnesium salts, of the following compounds: alkyl sulphates, alkyl ether sulphates, alkylamido ether sulphates, alkylarylpolyether sulphates, monoglyceride sulphates; alkylsulphonates, alkylamidesulphonates, alkylarylsulphonates, α-olefin sulphonates, paraffin sulphonates; alkylsulphosuccinates, alkyl ether sulphosuccinates, alkylamide sulphosuccinates; alkylsulphoacetates; acylsarcosinates; and acylglutamates, the alkyl and acyl groups of all these compounds containing from 6 to 24 carbon atoms and the aryl group preferably denoting a phenyl or benzyl group.

In the context of the present invention, it is also possible to use C₆-C₂₄ alkyl esters of polyglycoside carboxylic acids such as alkyl glucoside citrates, polyalkyl glycoside tartrates, and polyalkyl glycoside sulphosuccinates; alkylsulphosuccinimates, acylise-thionates and N-acyltaurates, the alkyl or acyl group of all these compounds containing from 12 to 20 carbon atoms. Among the anionic surfactants that may also be used, mention may also be made of acyllactylates in which the acyl group contains from 8 to 20 carbon atoms.

In addition, mention may also be made of alkyl-D-galactosideuronic acids and the salts thereof, and also of polyoxyalkylenated (C₆-C₂₄)alkyl ether carboxylic acids, polyoxyalkylenated (C₆-C₂₄)alkyl(C₆-C₂₄)aryl ether carboxylic acids, polyoxyalkylenated (C₆-C₂₄)alkylamido ether carboxylic acids and salts thereof, in particular those containing from 2 to 50 ethylene oxide groups, and mixtures thereof.

Among the above mentioned anionic surfactants that are preferably used according to the invention are (C₆-C₂₄)alkyl sulphates, (C₆-C₂₄)alkyl ether sulphates, (C₆-C₂₄)alkyl ether carboxylates and mixtures thereof, for example ammonium lauryl sulphate, sodium lauryl sulphate, magnesium lauryl sulphate, sodium lauryl ether sulphate, ammonium lauryl ether sulphate and magnesium lauryl ether sulphate.

The composition according to the present invention may comprise the anionic surfactants in an amount preferably of between 0.5% and 60% by weight and better still between 5% and 20% by weight, relative to the total weight of the composition.

The nonionic surfactants that may be used in the context of the present invention are, themselves also, compounds that are well known per se (see in particular in this respect “Handbook of Surfactants” by M. R. Porter, published by Blackie & Son (Glasgow and London), 1991, pp. 116-178). Thus, they can be chosen in particular from alcohols, α-diols, (C₁-C₂₀)alkyl phenols or polyethoxylated, polypropoxylated or polyglycerolated fatty acids, having a fatty chain containing, for example, 8 to 18 carbon atoms, it being possible for the number of ethylene oxide or propylene oxide groups to range in particular from 2 to 50 and for the number of glycerol groups to range in particular from 2 to 30. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; polyethoxylated fatty amines preferably having 2 to 30 mol of ethylene oxide; ethoxylated fatty acid esters of sorbitan having from 2 to 30 mol of ethylene oxide; fatty acid esters of sucrose, fatty acid esters of polyethylene glycol, (C₆-C₂₄) alkylpolyglucosides, N-(C₆-C₂₄)alkylglucamine derivatives, amine oxides such as (C₁₀-C₁₄)alkylamine oxides or N-(C₁₀-C₁₄)acylaminopropyl-morpholine oxides; and mixtures thereof.

Among the above mentioned nonionic surfactants that are preferably used are (C₆-C₂₄)alkylpolyglycosides, in particular decylpolyglucoside.

The amphoteric surfactants that are suitable for use in the present invention may especially be aliphatic secondary or tertiary amine derivatives, in which the aliphatic radical is a linear or branched chain containing 8 to 22 carbon atoms and containing at least one water-soluble anionic group (for example carboxylate, sulphonate, sulphate, phosphate or phosphonate); mention may also be made of (C₈-C₂₀)alkylbetaines, sulphobetaines, (C₈-C₂₀)alkylamido(C₆-C₈)alkylbetaines or (C₈-C₂₀)alkylamido(C₆-C₈)-alkylsulphobetaines; and mixtures thereof.

Among the amine derivatives that may be mentioned are the products sold under the name “Miranol®”, as described in patents U.S. Pat. No. 2,528,378 and U.S. Pat. No. 2,781,354 and classified in the CTFA dictionary, 3rd edition, 1982, under the names Amphocarboxyglycinate and Amphocarboxypropionate, and having the respective structures (1) and (2):

R₂—CONHCH₂CH₂—N⁺(R₃) (R₄) (CH₂COO⁻) (1)

in which:

R₂ represents an alkyl group derived from an acid R₂—COOH present in hydrolysed coconut oil, or a heptyl, nonyl or undecyl group,

R₃ represents a β-hydroxyethyl group, and

R₄ represents a carboxymethyl group; and

R₂′—CONHCH₂CH₂—N(B) (C) (2)

in which:

B represents —CH₂CH₂OX′,

C represents —(CH₂)_{z}—Y′, with z=1 or 2,

X′ represents the —CH₂CH₂—COOH group or a hydrogen atom,

Y′ represents —COOH or the —CH₂—CHOH—SO₃H group,

R₂′ represents the alkyl group of an acid R₂′—COOH present in coconut oil or in hydrolysed linseed oil, an alkyl group, especially a C₁₇ group and its isoform, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, mention may be made of the cocoamphodiacetate sold under the trade name Miranol® C2M concentrated by the company Rhodia.

Among the amphoteric surfactants that are preferably used are (C₈-C₂₀)alkylbetaines such as cocobetaine, (C₈-C₂₀)alkylamidoalkyl (C₆-C₈)betaines such as cocamidobetaine, and alkylamphodiacetates, for instance disodium cocoamphodiacetate, and mixtures thereof.

The composition according to the invention may also comprise one or more cationic surfactants that are well known per se, such as primary, secondary or tertiary fatty amine salts, optionally polyoxyalkylenated; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; or amine oxides of cationic nature.

The nonionic, amphoteric and cationic surfactants described above may be used alone or as mixtures and the amount thereof is between 0.1% and 30% by weight, preferably between 0.5% and 25% by weight and better still between 1% and 20% by weight, relative to the total weight of the composition.

The silicones that may be used as additives in the cosmetic compositions of the present invention are volatile or non-volatile, cyclic, linear or branched silicones, optionally modified with organic groups, having a viscosity from 5×10⁻⁶ to 2.5 m²/s at 25° C. and preferably 1×10⁻⁵ to 1 m²/s.

The silicones that may be used in accordance with the invention may be soluble or insoluble in the composition and in particular may be polyorganosiloxanes that are insoluble in the composition of the invention. They may be in the form of oils, waxes, resins or gums.

The organopolysiloxanes are defined in greater detail in Walter Noll's “Chemistry and Technology of Silicones” (1968), Academic Press. They can be volatile or non-volatile.

When they are volatile, the silicones are more particularly chosen from those having a boiling point of between 60° C. and 260° C., and even more particularly from:

(i) cyclic silicones containing from 3 to 7 and preferably from 4 to 5 silicon atoms. These are, for example, octamethylcyclotetrasiloxane sold in particular under the name “Volatile Silicone® 7207” by Union Carbide or “Silbione® 70045 V 2” by Rhodia, decamethylcyclopentasiloxane sold under the name “Volatile Silicone® 7158” by Union Carbide, and “Silbione® 70045 V 5” by Rhodia, and mixtures thereof.

Mention may also be made of cyclocopolymers of the dimethylsiloxanes/methylalkylsiloxane type, such as “Volatile Silicone® FZ 3109” sold by the company Union Carbide, with the chemical structure:

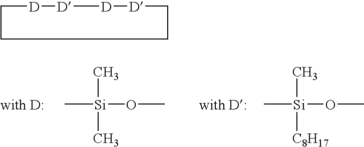

Mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is decamethyltetrasiloxane sold in particular under the name “SH 200” by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Non-volatile silicones, and more particularly polyalkylsiloxanes, polyarylsiloxanes, polyalkylarylsiloxanes, silicone gums and resins, polyorganosiloxanes modified with organofunctional groups, and mixtures thereof, are preferably used.

These silicones are more particularly chosen from polyalkylsiloxanes, among which mention may be made mainly of polydimethylsiloxanes containing trimethylsilyl end groups. The viscosity of the silicones is measured at 25° C. according to ASTM standard 445 Appendix C.

Among these polyalkylsiloxanes, mention may be made, in a non-limiting manner, of the following commercial products:

the Silbione® oils of the 47 and 70 047 series or the Mirasil® oils sold by Rhodia, such as, for example, the oil 70 047 V 500 000;

the oils of the Mirasil® series sold by the company Rhodia;

the oils of the 200 series from the company Dow Corning, such as DC200 with a viscosity of 60 000 mm²/s;

the Viscasil® oils from General Electric and certain oils of the SF series (SF 96, SF 18) from General Electric.

Mention may also be made of polydimethylsiloxanes containing dimethylsilanol end groups, known by the name Dimethiconol (CTFA), such as the oils of the 48 series from the company Rhodia.

In this category of polyalkylsiloxanes, mention may also be made of the products sold under the names “Abil Wax® 9800 and 9801” by the company Goldschmidt, which are poly (C₁-C₂₀)alkylsiloxanes.

The polyalkylarylsiloxanes are chosen particularly from linear and/or branched polydimethyl/methylphenylsiloxanes and polydimethyldiphenylsiloxanes with a viscosity of from 1×10⁻⁵ to 5×10⁻² m²/s at 25° C.

Among these polyalkylarylsiloxanes, mention may be made, by way of example, of the products sold under the following names:

the Silbione® oils of the 70 641 series from Rhodia;

the oils of the Rhodorsil® 70 633 and 763 series from Rhodia;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000;

certain oils of the SF series from General Electric, such as SF 1023, SF 1154, SF 1250 and SF 1265.

The silicone gums that can be used in accordance with the invention are, in particular, polydiorganosiloxanes having high number-average molecular masses of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Mention may be made more particularly of the following products:

polydimethylsiloxane

polydimethylsiloxane/methylvinylsiloxane gums,

polydimethylsiloxane/diphenylsiloxane,

polydimethylsiloxane/phenylmethylsiloxane,

polydimethylsiloxane/diphenylsiloxane/methylvinylsiloxane.

Products that can be used more particularly in accordance with the invention are mixtures such as:

mixtures formed from a polydimethylsiloxane hydroxylated at the end of the chain (referred to as dimethiconol according to the nomenclature in the CTFA dictionary) and from a cyclic polydimethylsiloxane referred to as cyclomethicone according to the nomenclature in the CTFA dictionary), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;

mixtures of two PDMSs of different viscosities, and more particularly of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product preferably contains 15% SE 30 gum and 85% SF 96 oil.

The organopolysiloxane resins that can be used in accordance with the invention are crosslinked siloxane systems containing the following units:

R₂SiO_{2/2}, R₃SiO_{1/2}, RSiO_{3/2} and SiO_{4/2}

in which R represents a hydrocarbon-based group containing 1 to 16 carbon atoms or a phenyl group. Among these products, those particularly preferred are the ones in which R denotes a C₁-C₄ lower alkyl radical, more particularly methyl, or a phenyl radical.

Among these resins, mention may be made of the product sold under the name “Dow Corning 593” or those sold under the names “Silicone Fluid SS 4230 and SS 4267” by the company General Electric, which are silicones of dimethyl/trimethyl siloxane structure.

Mention may also be made of the trimethyl siloxysilicate type resins sold in particular under the names “X22-4914, X21-5034 and X21-5037” by the company Shin-Etsu.

The organomodified silicones that can be used in accordance with the invention are silicones as defined above and containing in their structure one or more organofunctional groups attached via a hydrocarbon-based radical.

Among the organomodified silicones, mention may be made of polyorganosiloxanes comprising:

polyethylenoxy and/or polypropylenoxy groups optionally containing C₆-C₂₄ alkyl groups, such as the products known as dimethicone copolyol sold by the company Dow Corning under the name “DC 1248” or the Silwet® L 722, L 7500, L77, L 711 oils by the company Union Carbide and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;

thiol groups such as the products sold under the names “GP 72 A” and “GP 71” from Genesee;

alkoxylated groups such as the product sold under the name “Silicone Copolymer F-755” by SWS Silicones and Abil Wax® 2428, 2434 and 2440 by the company Goldschmidt;

hydroxylated groups such as the polyorganosiloxanes containing a hydroxyalkyl function, described in French patent application FR-A-8516334;

acyloxyalkyl groups such as, for example, the polyorganosiloxanes described in patent U.S. Pat. No. 4,957,732;

anionic groups of carboxylic type, such as, for example, in the products described in patent EP 186 507 from the company Chisso Corporation, or of alkylcarboxylic type, such as those present in the product X-22-3701E from the company Shin-Etsu; 2-hydroxyalkyl sulphonate; 2-hydroxyalkyl thiosulphate such as the products sold by the company Goldschmidt under the names “Abil® S201” and “Abil® S255”;

hydroxyacylamino groups, such as the polyorganosiloxanes described in patent application EP 342 834. Mention may be made, for example, of the product Q2-8413 from the company Dow Corning.

The silicones as described above may be used, alone or as a mixture, in an amount of between 0.01% and 20% by weight and preferably between 0.1% and 5% by weight.

The cosmetically acceptable aqueous medium may contain mineral or organic electrolytes.

The electrolytes used are preferably water-soluble mineral salts such as alkali metal, alkaline-earth metal or aluminium salts, hydrochloric acid, sulphuric acid or nitric acid salts, or alternatively organic acid salts such as alkali metal, alkaline-earth metal or aluminium carbonates, lactates, citrates or tartrates. The electrolytes that are particularly preferred are chosen from potassium sulphate, sodium sulphate, magnesium sulphate, calcium nitrate, magnesium nitrate, sodium chloride, potassium chloride, potassium carbonate, sodium carbonate and sodium citrate.

These electrolytes are preferably present in proportions ranging from 0.1% to 30% by weight and in particular from 1% to 10% by weight, relative to the total weight of the composition.

The pH of the aqueous compositions of the present invention is preferably set at a value of between 3 and 11 and in particular between 4 and 9.

### EXAMPLE 1

### Preparation of a Self-adhesive Polyurethane

The following monomers and solvents are introduced into a thermostatically regulated reactor equipped with a mechanical stirring system and a condenser:

1 mol of a mixture of diols, i.e. a mixture of N-methyldiethanolamine and poly(tetramethylene oxide) with a weight-average molar mass equal to 1400, the molar ratio of the N-methyldiethanolamine (NMDEA) to the poly(tetramethylene oxide) (PTMO) being equal to 2, and

an amount of methyl ethyl ketone (solvent) such that the concentration of diol monomers is equal to 75% by weight.

The mixture is heated with stirring to a temperature of 70° C., followed by dropwise addition with stirring, over a period of about 2 hours, of a small molar excess, i.e. 1.03 mol, of tetramethylxylylene diisocyanate (OCN—C(CH₃)₂-phenylene-C(CH₃)₂NCO) (TXDI).

During this addition, an increase in temperature up to the reflux point of the solvent is observed.

A sample is withdrawn at regular intervals and the IR absorption spectrum thereof is plotted to monitor the disappearance of the band corresponding to the isocyanate functions (2260 cm⁻¹).

When the absorption band for the —NCO functions no longer decreases, which is generally the case after about 5 hours, the reaction mixture is allowed to cool to room temperature and is then diluted with acetone to a polymer concentration of about 40% by weight.

20 ml of ethanol are then added to the mixture obtained so as to deactivate the residual —NCO functions, and stirring is continued at room temperature until all of the —NCO functions have disappeared, i.e. the IR absorption band at 2260 cm⁻¹.

A hydrochloric acid solution (2 mol/l) is added in an amount such that 100% of the amine groups are neutralized. The various organic solvents (methyl ethyl ketone, acetone and ethanol) are then removed by distillation under vacuum at a temperature of 40° C.

After removal of the organic phase, an amount of water sufficient to obtain a polymer concentration in the water of about 25% by weight is added to the aqueous polymer solution.

The polyurethane (TXDI/NMDEA/PTMO) thus obtained has a weight-average molar mass and a number-average molar mass, determined by gel permeation chromatography, equal to 70 900 and 43 800, respectively, which allows a polydispersity index of about 1.6 to be calculated.

### EXAMPLE 2

A shampoo having the composition below is prepared:

| Sodium lauryl ether sulphate containing | | |
|---|---|---|
| 2.2 mol of ethylene oxide | 12.5 g | (a.m.) |
| Cocoylbetaine | 2.5 g | (a.m.) |
| Polyurethane of Example 1 | 3 g | (a.m.) |
| pH regulator | qs pH 7 | |
| Demineralized water | 100 | g |

This composition gives dry hair a styling effect that is reflected by good shapeability.

1. Cationic or amphoteric polyurethanes bearing at least one tertiary or quaternary amine function, characterized in that they have a self-adhesion value, expressed by the maximum tensile force (Fₘₐₓ (in N)), of greater or equal to 11 N.

2. Polyurethanes according to claim 1, characterized in that they comprise
(a) units derived from one or more tertiary or quaternary amines comprising two reactive functions containing labile hydrogen,
(b) units derived from one or more nonionic polymers comprising two reactive functions containing labile hydrogen, and
(c) units derived from one or more diisocyanates.

3. Polyurethanes according to claim 2, characterized in that the units (a) are derived from one or more amines corresponding to one of the following six formulae: 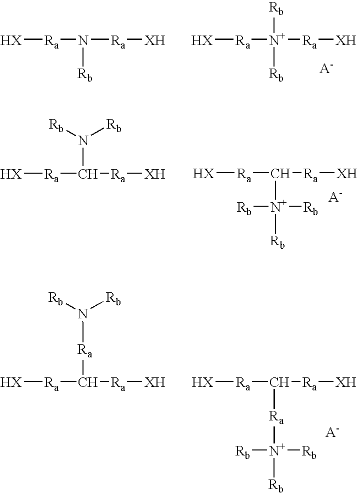
in which
each radical Rₐ represents, independently, a linear or branched C₁₋₆ alkylene, C₃₋₆ cycloalkylene or arylene group, all possibly being substituted with one or more halogen atoms and comprising one or more hetero atoms chosen from O, N, P and S,
each R_{b} represents, independently, a C₁₋₆ alkyl, C₃₋₆ cycloalkyl or aryl group, all possibly being substituted with one or more halogen atoms and comprising one or more hetero atoms chosen from O, N, P and S,
each radical X represents, independently, an oxygen or sulphur atom or a group NH or NR_{c}, in which R_{c} represents a C₁₋₆ alkyl group and
A⁻ represents a physiologically acceptable counterion.

4. Polyurethanes according to claim 3, characterized in that the units (a) are N-methyldiethanolamine or N-tert-butyldiethanolamine.

5. Polyurethanes according to any one of claims 2 to 4, characterized in that the units (a) containing a tertiary amine function are partially or totally neutralized with mineral or organic acids.

6. Polyurethanes according to any one of claims 2 to 5, characterized in that the polymers forming the units (b) are chosen from polyethers, polyesters, polysiloxanes, copolymers of ethylene and of butylene, polycarbonates or fluorinated polymers, all having a glass transition temperature of less than 0° C.

7. Polyurethanes according to claim 6, characterized in that the polymers forming the units (b) have a weight-average molar mass of between 400 and 10 000 and preferably between 500 and 5 000.

8. Polyurethanes according to claim 6 or 7, characterized in that the units (b) are poly(tetramethylene oxide) units.

9. Polyurethanes according to any one of claims 2 to 8, characterized in that the units (c) are diisocyanates chosen from tetramethylxylylene diisocyanate, methylenediphenyl diisocyanate, methylenecyclohexane diisocyanate, isophorone diisocyanate, toluene diisocyanate, naphthalene diisocyanate, butane diisocyanate and hexyl diisocyanate.

10. Polyurethanes according to any one of the preceding claims, characterized in that the molar ratio of the units (a) to the units (b) is between 0.01 and 50, preferably between 0.1 and 6, better still between 0.2 and 5 and ideally between 0.3 and 5.

11. Polyurethanes according to any one of the preceding claims, characterized in that they also comprise one or more anionic units (d).

12. Polyurethanes according to claim 11, characterized in that the anionic units (d) are derived from carboxylic acids or sulphonic acids comprising two functions containing labile hydrogen.

13. Cationic polyurethanes according to any one of the preceding claims, characterized in that the nonionic polymer(s) forming the units (b) have a glass transition temperature (Tg), determined by differential scanning calorimetry (DSC), of less than 0° C., preferably less than −5° C. and in particular less than −10° C.

14. Cationic polyurethanes according to any one of the preceding claims, characterized in that they have at least one glass transition temperature (Tg), determined by differential scanning calorimetry, of less than 20° C., preferably less than 0° C. and in particular less than −20° C.

15. Cosmetic composition containing, in a cosmetically acceptable aqueous medium, at least one self-adhesive cationic or amphoteric polyurethane according to any one of the preceding claims.

16. Cosmetic composition according to claim 15, characterized in that it contains from 0.01% to 40%, preferably from 0.05% to 20% and in particular from 0.1% to 20% by weight of cationic polyurethanes according to one of claims 1 to 13.

17. Cosmetic composition according to either of claims 15 and 16, characterized in that it is a care, conditioning, makeup or fixing composition for human keratin materials, in particular for the hair and the eyelashes.

18. Cosmetic composition according to claim 17, characterized in that it is a styling composition.

19. Cosmetic composition according to claim 18, characterized in that it is a rinse-out styling composition.

20. Cosmetic composition according to any one of claims 15 to 19, characterized in that it contains additives chosen from surfactants, anionic, amphoteric, zwitterionic or nonionic polymers, cationic polymers other than the cationic polyurethanes according to claims 1 to 14, nacreous agents and/or opacifiers, organic solvents, fragrances, mineral, plant and/or synthetic oils, fatty acid esters, pigments and colorants, silicones, mineral or organic particles, pH stabilizers, preserving agents and UV absorbers.

21. Cosmetic composition according to claim 20, characterized in that the surfactants are chosen from anionic, nonionic, amphoteric and cationic surfactants and mixtures thereof.

22. Cosmetic composition according to claim 21, characterized in that the anionic surfactants are present in a proportion of from 0.5% to 60% by weight and preferably from 5% to 20% by weight, and in that the nonionic, amphoteric and cationic surfactants are present in a proportion of from 0.1% to 30% by weight and preferably from 0.5% to 25% by weight, relative to the total weight of the composition.

23. Cosmetic composition according to claim 20, characterized in that the silicones are chosen from volatile or non-volatile, cyclic, linear or branched silicones, optionally modified with organic groups.

24. Cosmetic composition according to claim 23, characterized in that the silicones are present in a proportion of from 0.01% to 20% by weight and preferably from 0.1% to 5% by weight.

25. Cosmetic use of the self-adhesive cationic polyurethanes according to any one of claims 1 to 14.

26. Process for treating keratin materials, comprising the application of a cosmetic composition according to any one of claims 15 to 24 to the keratin materials to be treated.

27. Styling process comprising the application of a cosmetic composition according to any one of claims 15 to 24 to the hair, rinsing the hair and then shaping and drying the rinsed hair.
